(19) United States
(12) Patent Application Publication
(10) Pub. No.: US 2018/0208975 A1
(43) Pub. Date: Jul. 26, 2018

(54) ASSAY FOR SIMULTANEOUS GENOMIC AND PROTEOMIC ANALYSIS

(71) Applicant: MERCK SHARP & DOHME CORP., Rahway, NJ (US)

(72) Inventors: Vanessa Marie Peterson, Jamaica Plain, MA (US); Joel Albert Klappenbach, Sudbury, MA (US)

(73) Assignee: MERCK SHARP & DOHME CORP., Rahway, NJ (US)

(21) Appl. No.: 15/873,331

(22) Filed: Jan. 17, 2018

Related U.S. Application Data
(60) Provisional application No. 62/448,613, filed on Jan. 20, 2017.

(51) Int. Cl. C12Q 1/6811; C12Q 1/6806; C12Q 1/6837; C12Q 1/6851; C12Q 1/686; C12Q 1/6876

(52) U.S. Cl. CPC C12Q 1/6811; C12Q 1/6806; C12Q 1/6837; C12Q 1/6851; C12Q 2600/16; C12Q 1/6876; C12Q 2600/158; C12Q 2600/166; C12Q 1/686

(57) ABSTRACT

The present invention is directed to a biochemical assay for simultaneous genomic and proteomic analysis.

### BACKGROUND OF THE INVENTION

Currently, clinical samples especially in the immuno-oncology area are difficult to obtain, heterogeneous in cellular content, and typically very small in size. Often times, for a given sample genomic profiling, proteomic analysis, immune profiling, biomarker monitoring etc. is needed. Unfortunately, clinical samples are typically limiting and there is insufficient material to perform both proteomic and genomic profiling. There are many post translational modifications that occur to proteins such as phosphorylation, ubiquitination, glycosalation, etc. that play an important role in regulating different cellular signaling pathways. There is a need for assays that enables quantification of an unlimited number of proteins and RNA species at the single-cell level.

Proof of concept of labeling cellular proteins with antibodies attached to DNA has been previously demonstrated using the NanoString platform as a readout (Ullal & Peterson, Sci. Transl. Med. 2014, 6(219)). This hybridization based method has limitations on multiplexing capabilities (<8400) and is not conducive for a single-cell high throughput platform (>1000's of cells).

Currently flow cytometry is the goldstandard in multiparametric cellular analysis and is limited to a maximum of ˜18 markers. Mass cytometry was recently (Bandura, Anal. Chem, 2009, 81(16):6813-6822) developed to increase the number of parameters that could be measured by conjugating elemental isotopes to antibodies (detected by mass spectroscopy) rather than fluorophores (photon detection). However, the increase was a modest two-fold (˜37 markers) and since the cells are ionized in the process it does not allow for any further downstream analysis to be conducted such as RNA-sequencing.

### SUMMARY OF THE INVENTION

The invention is a biochemical assay for simultaneous genomic and proteomic analysis. In one embodiment, it is used for single-cell resolution using sequencing as a readout. This method enables quantification of an unlimited number of proteins and RNA species in thousands of single-cells. In addition, this assay can also be applied to small subpopulations of cells or cell lysates for high throughput screening. The assay is designed to efficiently capture and analyze cells from small clinical biopsies where cell number is limited.

### BRIEF DESCRIPTION OF THE DRAWINGS

FIG. 1: Diagram of single cell assay for protein and RNA expression profiling.

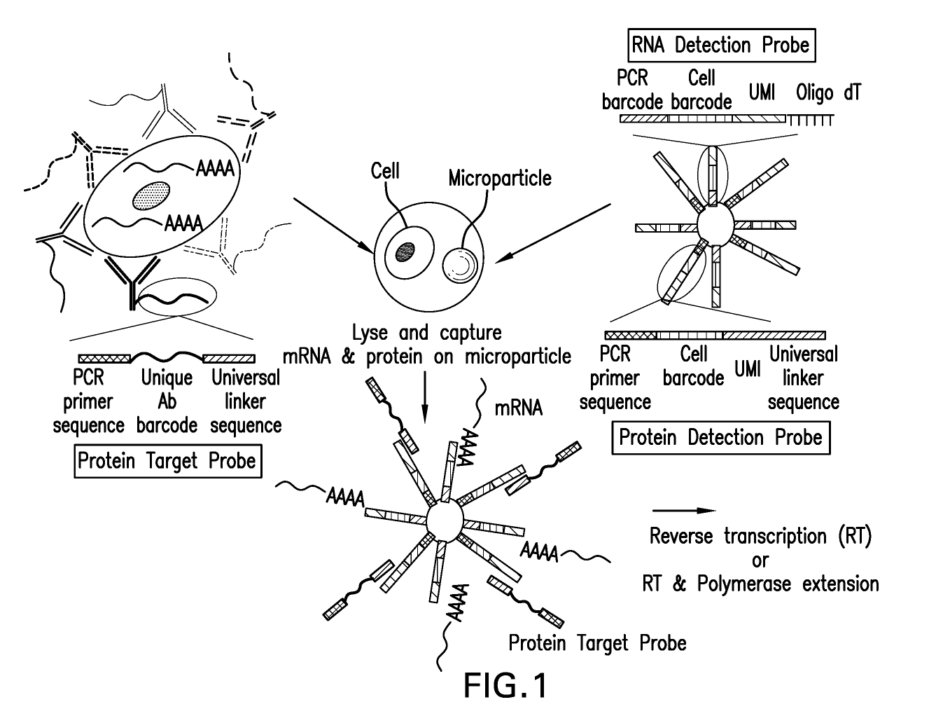

FIG. 2: Schematic of modified PEA assay for protein detection part of assay. Oligo A comprises 5′ to 3′ universal primers (Read 1 or Read 2 illumina sequences), Ab barcode A, Oligo A and oligoB complementary sequence. Oligo B comprises 5′ to 3′ extension product and protein detection probe complementary sequence, Ab barcode B, Oligo A and oligoB complementary sequence. The protein detection probe comprises from 5′ to 3′ universal primers (Read 1 or Read 2 illumina sequences), cell barcode+UMI, and extension product and protein detection probe complementary sequence. The protein analyte sequence comprises from 5′ to 3′ universal primers (Read 1 or Read 2 illumina sequences), cell barcode+UMI, extension product and protein detection probe complementary sequence, Ab barcode B, Oligo A and oligoB complementary sequence, Ab barcode A, universal primers (Read 1 or Read 2 illumina sequences).

FIG. 3: Alternative schematic of modified PEAS assay for protein detection part of assay. Oligo A comprises 5′ to 3′ universal hybridization sequence A and Ab barcode A. Oligo B comprises 5′ to 3′, universal primers (Read 1 or Read 2 illumina sequences), Ab barcode B, universal hybridization sequence B. The protein detection probe comprises from 5′ to 3′ universal primers (Read 1 or Read 2 illumina sequences), cell barcode+UMI, Ab barcode A, universal hybridization sequence A, universal hybridization sequence B. The protein analyte sequence comprises from 5′ to 3′ universal primers (Read 1 or Read 2 illumina sequences), cell barcode+UMI, Ab barcode A, universal hybridization sequence A, universal hybridization sequence B, Ab barcode B, universal primers (Read 1 or Read 2 illumina sequences).

FIG. 4: Schematic of microparticle with detection probes.

FIG. 5A-B: A) capturing single cells and microparticles via emulsion based droplets. B) capturing single cells and microparticles via an array of microwells.

### DETAILED DESCRIPTION

The assay of the invention enables both proteomic and genomic profiling through combined analysis of RNA expression and protein sequencing. The combined platform will eliminate the challenge of comparing data from different technologies with different outputs or from different aliquots of sample. This assay's ability to achieve single-cell resolution provides an unbiased view of the heterogeneity within the tumor microenvironment and enables identification of rare cell types, immune cells driving response, or anergic cell types associated with lack of tumor response to immunotherapy. Cellular heterogeneity could then be related to therapeutic response and permit the discovery of mechanisms of resistance and identify new biomarkers to predict therapeutic response. This assay in addition to being applied to single cells, a plurality of cell or a cell lysate can also be extended to high throughput cell-based screens of small molecules and antibodies for drug discovery.

The present invention provides a method for detecting a plurality of target proteins and mRNA in a cell or cell lysate comprising:

a. contacting a cell or cell lysate with
1) a plurality of protein target probes, wherein each target probe in the plurality comprises:
i. a protein-binding molecule;
ii. a target nucleotide sequence comprising a primer for a polymerase chain reaction (PCR), a protein identification sequence that identifies the protein-binding molecule, a linker sequence that hybridizes to a sequence in the detection probe;
iii. a linker between the protein-binding molecule and the target nucleotide sequence; and
2) a plurality of microparticles comprising a plurality of detection probes comprising:
i. a protein detection nucleotide sequence comprising a primer for a polymerase chain reaction (PCR), a cell identification sequence that identifies the cell, a unique molecular identifier (UMI), and a complimentary linker sequence that hybridizes to the linker sequence in the target probe;
ii. an mRNA detection nucleotide sequence comprising a primer for a polymerase chain reaction (PCR), a cell identification sequence that identifies the cell, a unique molecular identifier, and polydT;
b. allowing the target nucleotide sequence to hybridize to the protein detection nucleotide sequence and the mRNA to hybridize to the mRNA detection nucleotide sequence in the microparticle;
c. conducting reverse transcription and/or polymerase extension to generate
i. a first analyte sequence comprising the primer, cell identification sequence, unique molecular identifier, and complimentary linker sequence in the detection probe; and protein identification sequence and the primer from the protein target probe;
ii. a second analyte sequence comprising the primer, cell identification sequence, unique molecular identifier, the polydT in the detection probe; and cDNA of the mRNA;
d. enriching the analyte sequences from unbound target and detection nucleotide sequences in the sample;
e. detecting signals from the analyte sequences based on PCR amplification and sequencing, wherein the signals are distinguishable for each protein and mRNA.

In another aspect of the invention is a method for detecting a plurality of target proteins and mRNA in a single cell:

a. contacting a single cell with a plurality of protein target probes, wherein each target probe in the plurality comprises:
1) a plurality of protein target probes, wherein each target probe in the plurality comprises:
i. a protein-binding molecule;
ii. a target nucleotide sequence comprising a primer for a polymerase chain reaction (PCR), a protein identification sequence that identifies the protein-binding molecule, a linker sequence that hybridizes to a sequence in the detection probe;
iii. a linker between the protein-binding molecule and the target nucleotide sequence; and
b. forming an emulsion droplet or microwell comprising the single cell and a microparticle comprising a plurality of detection probes comprising:
i. a protein detection nucleotide sequence comprising a primer for a polymerase chain reaction (PCR), a cell identification sequence that identifies the cell, a unique molecular identifier, and a complimentary linker sequence that hybridizes to the linker sequence in the target probe;
ii. an mRNA detection nucleotide sequence comprising a primer for a polymerase chain reaction (PCR), an cell identification sequence that identifies the cell, a unique molecular identifier, and polydT;
c. allowing the target nucleotide sequence to hybridize to the protein detection nucleotide sequence and the mRNA to hybridize to the mRNA detection nucleotide sequence in the microparticle;
d. conducting reverse transcription and/or polymerase extension to generate
i. a first analyte sequence comprising the primer, cell identification sequence, unique molecular identifier, and complimentary linker sequence in the detection probe; and protein identification sequence and the primer from the protein target probe;
ii. a second analyte sequence comprising the primer, cell identification sequence, unique molecular identifier, and polydT in the detection probe; and cDNA of the mRNA;
e. enriching the analyte sequences from unbound target and detection nucleotide sequences in the sample;
f. detecting signals from the analyte sequences based on PCR amplification and sequencing, wherein the signals are distinguishable for each protein and mRNA.

In one embodiment, the single cell is encapsulated with the microparticle in a droplet. This can be achieved by a microfluidic device. (Macosko et. al. Cell 2015, 161(5): 1202-1214; Klein et. al. Cell 2015, 161(5): 1187-1201) where two aqueous solutions are flowed across an oil channel to form more than 100,000 nanoliter sized droplets per minute. One flow contains the barcoded microparticles suspended in lysis buffer; the other flow contains a cell suspension. The number of droplets created greatly exceeds the number of beads or cells injected, so that a droplet will generally contain zero or one cell, and zero or one bead. The emulsion based technology will enable deep single-cell profiling in a high throughput manner—for example 100,000 of individual cells from samples where cell number is limited and do not have enough material for standard methods of proteomic measurements such as flow cytometry or mass cytometry. Emulsion based microfluidic technologies have been previously demonstrated with regards to RNA sequencing as a very efficient way to generate data in an inexpensive and high throughput manner (Macosko et. al. Cell 2015, 161(5): 1202-1214; Klein et. al. Cell 2015, 161(5): 1187-1201.). For the first time, in the present invention, the emulsion based technology is applied to measuring protein in addition to RNA expression. In another embodiment, the single cell and microparticle can be captured in a microwell.

In one embodiment, the protein target probes are nucleotide sequence barcoded antibodies. Nucleotide sequence barcodes conjugated to antibodies permit a nearly unlimited number of molecular tags and proteomic multiplexing capability. The linker between the protein-binding molecule and protein target probe can be cleavable and include but are not limited to hydrolyzable linkers, redox cleavable linkers, phosphate-based cleavable linkers, acid cleavable linkers, ester-based cleavable linkers, peptide-based cleavable linkers, photocleavable linkers, and any combinations thereof. In one embodiment, the target nucleotide sequence is attached to the antibody with a photocleavable linker. In one embodiment, the target nucleotide sequence is DNA. After the antibody binds to the protein in the cell, and the cell is encapsulated with the microparticle in a droplet or microwell, the cleavable linker releases the unique target nucleotide sequence, which hybridizes to the detection probe. In one embodiment, the DNA barcode comprise a primer for PCR amplification, an identification sequence to identify the protein, and a universal linker sequence that hybridizes to a detection probe on the microparticle. In one embodiment, the invention employs conjugating an 8-20, or 10 bp nucleotide sequence to identify the protein which will increase the multiplexing capability to ˜1×10⁶.

In another embodiment, the protein-binding molecule is an aptamer. See Gold et al., PLos ONE, 5(12):e15004.

The microparticle can be attached to the detection probes covalently or non-covalently. In one embodiment, the microparticle comprises a polymer bead, magnetic bead, hydrogel microsphere, or resin that has the protein and mRNA detection nucleotide sequences bound to or encapsulated in the microparticle. In one embodiment, the microparticle comprises biotinylated detection probes attached to streptavidin beads. In another embodiment, the hydrogel is a polyacrylamide mesh attached to the detection probes via a photocleavable linker. Typically, each bead contains between 10⁶ to 10¹⁰ probes. The protein detection nucleotide sequence comprises a primer for a polymerase chain reaction (PCR), a cell identification sequence that identifies the cell, a unique molecular identifier, and a complimentary linker sequence that hybridizes to the linker sequence in the target probe. In one embodiment, the complimentary linker sequence that hybridizes to the target probe is universal. In a further embodiment, the primer in the protein and mRNA detection nucleotide sequence comprises a common sequence to enable PCR amplification. Typically, the sequence that identifies the cell in the detection probe is identical across the surface of any one bead, but different from the sequence on other beads.

The mRNA detection nucleotide sequence on the microparticle comprises a primer for a polymerase chain reaction (PCR), a cell identification sequence that identifies the cell, a unique molecular identifier, and polydT. The polydT sequence hybridizes to the polyA tail of the mRNA. In one embodiment, the polydT is 10-40, or 30 nucleotides in length.

The unique molecular identifiers (UMI) used in the detection probes of the microparticle provide absolute quantification of proteins and mRNA molecules at the single-cell level. Kivioja et al. Nat. Methods 9, 72-74. The UMIs are different for each mRNA or protein molecule. UMI's correct for any bias that may occur during the amplification steps needed for single-cell level detection. The UMI can be 4-20, 10-15 or 12 nucleotides in length utilizing A, G, C or T. For example, four DNA bases can be subject to 12 rounds of degenerate synthesis, such that each individual probe receives one of 412 possible UMIs.

Following droplet or microwell formation, the single cell is lysed to release the mRNA and protein target probes to hybridize to the detection probes on the microparticle. The droplets can be broken by adding a reagent to destabilize the oil-water interface, and the microparticles are collected and washed. Through reverse transcription or DNA polymerase chain extension, the target nucleotide sequence-detection probe complex will generate a first analyte sequence. Through reverse transcription, the mRNA-detection probe complex will generate a second analyte sequence. In one embodiment, template switching is used to introduce a PCR primer downstream of the synthesized cDNA. See Zhu et al., Biotechniques 30, 892-897 (2001). In another embodiment, a random-primed Klenow extension can be used to facilitate PCR amplification. See Derti et al., Genome Research, 22:1173-1183 (2012). The analyte sequences are then PCR amplified, quantified, sequenced and quantified. Individual UMIs are counted for each gene or protein in each cell.

The PCR amplification can be based on quantitative polymerase chain reaction (PCR) or multiplexed PCR. The sequencing can be based on for example, DNA sequencing, RNA sequencing, next-generation sequencing such as massively parallel signature sequencing (MPSS), Illumina (Solexa) sequencing, SOLiD sequencing, ion semiconductor sequencing, DNA nanoball sequencing, Heliscope single molecule sequencing, single molecule real time (SMRT) sequencing, nanopore DNA sequencing, sequencing by hybridization, sequencing with mass spectrometry, microfiuidic Sanger sequencing, microscopy-based sequencing techniques, RNA polymerase (RNAP) sequencing, or any combinations thereof.

The present invention also provides a kit for multiplexed detection of a plurality of proteins and mRNA from a sample comprising

1) a plurality of protein target probes, wherein each target probe in the plurality comprises:
i. a protein-binding molecule;
ii. a target nucleotide sequence comprising a primer for a polymerase chain reaction (PCR), a protein identification sequence that identifies the protein-binding molecule, a linker sequence that hybridizes to a sequence in the detection probe;
iii. a linker between the protein-binding molecule and the target nucleotide sequence; and
2) a plurality of microparticles comprising a plurality of detection probes comprising:
i. a protein detection nucleotide sequence comprising a primer for a polymerase chain reaction (PCR), a cell identification sequence that identifies the cell, a unique molecular identifier, and a complimentary linker sequence that hybridizes to the linker sequence in the target probe; and
ii. an mRNA detection nucleotide sequence comprising a primer for a polymerase chain reaction (PCR), a cell identification sequence that identifies the cell, a unique molecular identifier, and polydT.

### EXAMPLES

### Example 1: Synthesis of Protein Detection Probes

Antibodies are conjugated to nucleotides via commercially available kits such as Thunder-link by Innova Biosciences (Lundberg et al., 2011a) and the antibody-oligonucleotide all-in-one kit by Solulink. Solulink's technology is based on the use of two complementary heterobifunctional linkers. It uses a two-step process to prepare an antibody-oligonucleotide. In the first step, an amine-modified 20 to 60 mer oligonucleotide is modified using an excess of Sulfo-S-4FB linker or by solid phase oligonucleotide synthesis using 4FB-phosphoramidite (Kasai et al., 2012). This reactive NHS-ester incorporates a 4FB (aromatic aldehyde functional group, formylbenzamide) at the desired terminus (5′ or 3′ end) of the oligonucleotide. In the second step, polyclonal or monoclonal antibodies are modified with S-HyNic linker. This NHS-ester reacts with lysine residues on the antibody incorporating HyNic functional groups (hydrazino-nicotinamide). The HyNic-modified antibody is then reacted with 4FB-modified oligonucleotide in the presence of an aniline catalyst (Dirksen and Dawson, 2008; Dirksen et al., 2006a; Dirksen et al., 2006b) to yield a stable bis-arylhydrazone mediated conjugate. This is followed by magnetic-affinity, solid phase purification to remove excess 4FB-oligonucleotide from antibody-oligonucleotide conjugates. An SDS-PAGE gel is then used to determine the amount of un-conjugated antibody, or unconjugated oligonucleotide present in the purified conjugate. To make this a cleavable linker, sulfo-SS-4FB is used instead of sulfo-S-4FB to modify the oligonucleotide. This sulfo-SS-4FB disulfide bond is cleaved later in subsequent steps with DTT to release the oligonucleotide from the antibody.

Alternatively, antibodies are conjugated using a photocleavable bifunctional linker (amine to thiol group) reacting with the amine groups on the antibodies. Thiol-modified nucleotides are reduced with dithiothreitol (DTT). The reduced nucleotides are then purified and reacted with the maleimide-activated antibodies. Antibody-nucleotide conjugates are then purified from excess free nucleotide. (Ullal et al., 2014).

Alternatively, antibodies are reacted with an amine to sulfhydryl linker, sulfosuccinimidyl 6-[3_(2-pyridyldithio)-propionamido] hexanoate (sulfo-LC-SPDP, Thermo Scientific) which has a disulfide bond which in later steps is cleaved with DTT to release the nucleotide from the antibody. During the antibody reaction, a thiolyated nucleotide is reduced with DTT. At the end of the reactions, excess sulfo-LC-SPDP is removed from the antibody and excess DTT is removed from the nucleotide. The antibody is then reacted with the reduced thiolyated nucleotide and the final antibody-nucleotide conjugate is purified from excess nucleotide (Ullal et al., 2014).

Alternatively, a copper free click reaction based on strain promoted alkyne-azide cycloaddition (SPACC) (Agasti et al., 2012) (Gong et al., 2016) is used to modify biomolecules. Dibenzocyclooctyne (DBCO) is reacted with azide-functionalized molecules under physiological conditions having no adverse effects on macro-biomolecules such as antibodies. Initially, DBCO-PEG5-NHS is reacted to the amine groups on the antibody. After the reaction, the free DBCO-PEG5NHS is removed using Amicon Ultra 0.5 NMWL 50 kDa Centrifugal Filters. Inclusion of the PEG5 linker increases the water solubility of the DBCO moiety and introduces a spacer between the bulk antibody and the oligonucleotide. To determine the number of DBCO molecules on the antibody, the absorbance at 309 and 280 nm is measured. The molar concentrations of DBCO and antibody is determined using their respective molar extinction coefficient (12 000 M-1 cm-1 for DBCO at 309 nm and 204 000 M-1 cm-1 for antibody at 280 nm). The number of DBCO molecules per antibody is calculated by dividing the molar concentration of DBCO by the molar concentration of antibody. The DBCO-functionalized antibody is then reacted with the azide-modified oligonucleotide (conducted at 4° C. for 16-18 hr). The unreacted oligonucleotides are removed using the Amicon ultra 0.5 NMWL 100 kDA Centrifugal Filter. The oligonucleotide-conjugated antibody is analyzed on the Agilent 2100 BioAnalyzer using the protein 230 kit following the manufacturer's instructions. After electrophoresis and image acquisition the band sizes in the gel image indicate the molecular weights of the antibodies, and the amount of un-conjugated antibody or oligonucleotide present in the purified conjugate is determined (Gong et al., 2016).

Alternatively, heterobifunctional cross-linkers, such as succinimidyl 4-hydrazinonicotinate acetone hydrazone (SANH) (Mocanu et al., 2011) succinimidyl 4-(N-maleimidomethyl) cyclohexane-1-carboxylate (SMCC) (Soderberg et al., 2006), or sulfo-SMCC (Assarsson et al., 2014) are used to introduce a bridge between the oligonucleotide and the antibody. For example, monoclonal or polyclonal antibodies (pAb) are resuspended at 1 mg/mL in PBS and reacted with Sulfo-succinimidyl-4-(N-maleimidomethyl) cyclohexane-1-caroxylate (Sulfo-SMCC, Thermo Scientific). The sulfo-SMCC conjugated antibodies are purified from excess sulfo-SMCC via Zeba columns. Oligonucleotides are synthesized with a thiol modification and are reduced with DTT. Excess DTT is removed using two consecutive 7 kDa Zeba plates equilibrated with 100 mM phosphate buffer. The SMCC-treated antibodies are mixed with the DTT treated oligonucleotides at 10× molar excess of oligonucleotide to Ab. The antibody oligonucleotide conjugates are purified using pre-wet Slide-A-Lyzer Mini 7 MWCO dialysis cups (Thermo Scientific) and dialyzed in 1 L PBS with 5 mM EDTA at 4° C. for two days with one buffer exchange to PBS. The antibody-oligonucleotide conjugates are diluted to 75 mg/mL in a PBS buffer containing 4 mM EDTA, 35 mg/mL ssDNA (Sigma-Aldrich), 0.1% fish gelatin, and 20 mM Tris HCl, and stored at 4° C. (Assarsson et al., 2014).

Alternatively, instead of using antibodies as the molecule to bind proteins other biomolecules such as aptamers are used. Aptamers are short single-stranded oligonucleotides, which fold into intricate molecular structures that bind with high affinity and specificity to proteins, peptides, and small molecules. (Brody and Gold, 2000; Famulok et al., 2007; Gold, 1995). There are many examples of high affinity RNA and DNA aptamers selected against human proteins (Gold, 1995). Aptamers are selected in vitro from enormously large libraries of randomized sequences by the process of Systematic Evolution of Ligands by EXponential enrichment (SELEX) (Ellington and Szostak, 1990; Tuerk and Gold, 1990). A new class of aptamers, the Slow Off-rate Modified Aptamer (SOMAmer) are used for difficult protein targets for which standard RNA and DNA SELEX do not yield high affinity aptamers. Nucleotides are chemically modified with protein-like function groups to expand chemical diversity. Also, aptamers with slow dissociation rates are selected for which allows selective disruption of non-specific binding interactions with a polyanionic competitor. SOMAmers enable selection for almost any protein target (Gold, 1995). A universal protein linker nucleotide sequence (10-30 bp) is added to the SOMAmer or aptamer sequence to enable binding to the protein detection probe with an identification nucleotide sequence that identifies the single cell or sample, a unique molecular identifier, universal primer sequence, a complementary universal protein linker nucleotide sequence. The two oligonucleotides (one from protein target probe and the other from the protein detection probe) contain a complementary region (universal protein linker) that allows annealing and extension by DNA synthesis enzymes or reverse transcriptase to form a DNA template that is subsequently amplified and sequenced.

### Example 2: Labeling Protein Molecules with Target Probes

Protein molecules on the cell surface of living cells are labeled with antibodies conjugated to oligonucleotides or aptamers. To label intracellular protein molecules the cells are fixed and permeabilized. Then antibodies conjugated to oligonucleotides or aptamers are added. Cells are centrifuged and supernatant with excess unbound antibodies or aptamers is removed. Washes are conducted to further remove unbound antibodies and aptamers. Cells are isolated and protein detection probes are added that will bind to the protein target probes.

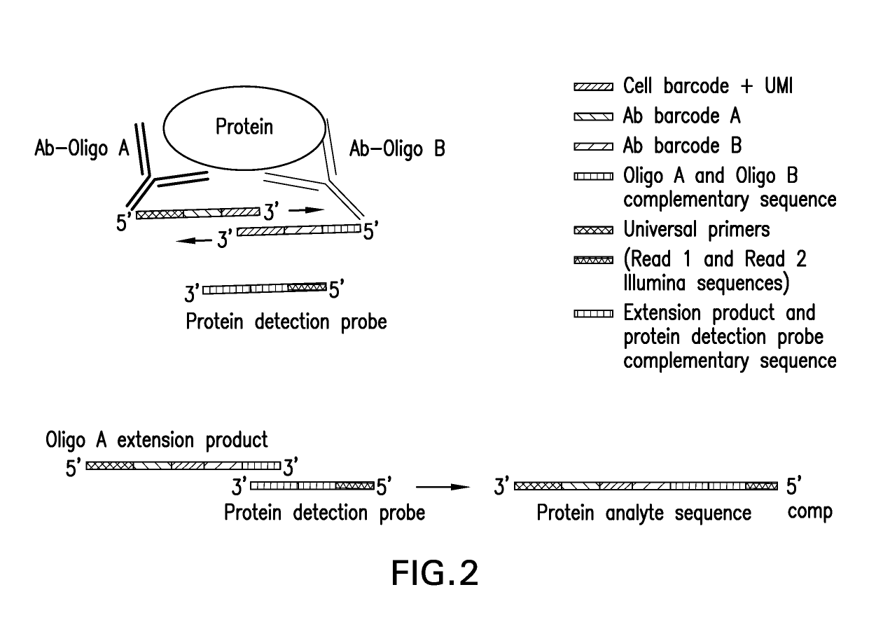

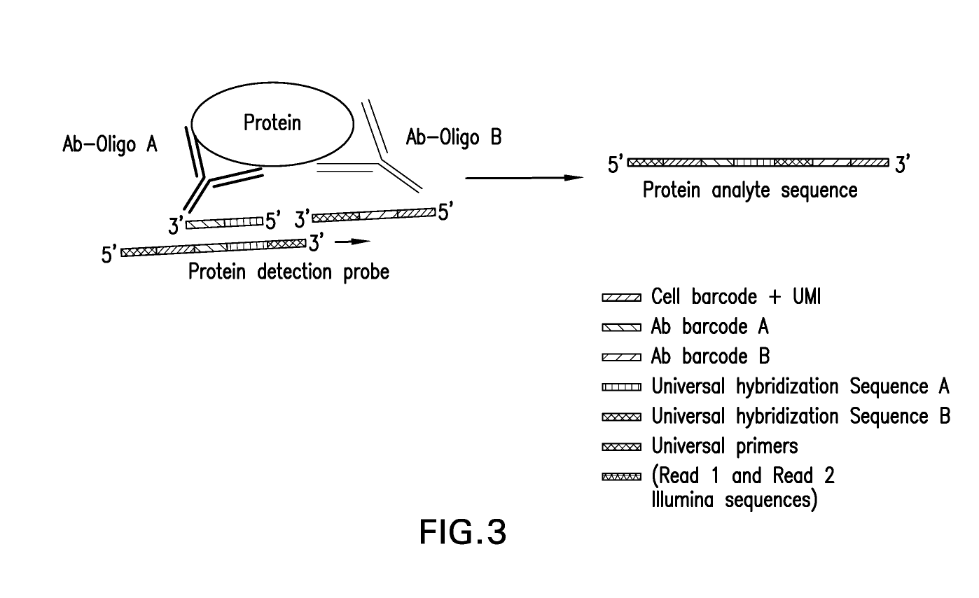

Alternatively, cells are lysed and extracellular and intracellular protein molecules are in solution. For each protein molecule of interest two antibodies, each conjugated with its own oligonucleotide with a complementary sequence to each other is added. A signal is produced only when the two antibodies bind to the same protein molecule and bring the two oligonucleotides in proximity, and form a DNA template through ligation or extension reaction (Fredriksson et al., 2002; Lundberg et al., 2011a; Lundberg et al., 2011b; Soderberg et al., 2006). One advantage of using two antibody binders is that the excess antibodies in solution will not produce signal because they are not in proximity to their respective partners. As a result, a washing step is no longer needed which offers many advantages for assay development. This hybridization based method has been called oligonucleotide extension reaction (OER)(Gong et al., 2016) or proximity extension assay (PEA). By attaching oligonucleotides with distinct sequences to different antibodies and converting the protein level to surrogate DNA level enables multiplex protein detection. The oligonucleotides on these two antibodies contain a complementary sequence to each other and a universal priming sequence. The detection probe is designed with a complementary sequence to the oligonucleotide after the extension reaction (FIG. 2) as well as with the nucleotide sequence specific for the cell or well, the unique molecular identifier sequence, and a universal priming sequence. Alternatively, the oligonucleotides on both antibodies do not have a complementary sequence to each other. However, the detection probe is designed with a complementary sequence to part of oligonucleotide A and part of oligonucleotide B (FIG. 3). When the two oligonucleotides are in proximity to each other the detection probe hybridizes to both of them and act as an extension primer that enables an extension reaction (Lundberg, 2011). The extended nucleotide with the target and detection probe sequence is subsequently amplified and Next-generation sequencing (NGS) methods are used as a readout.

### Example 3: Labeling RNA Molecules with Detection Probes

Detection probes containing a poly dT sequence capture the polyA tail of the mRNA. The mRNA is reverse transcribed to cDNA and a template switching oligonucleotide (TSO) is used to introduce a PCR handle downstream of the synthesized cDNA (Zhu et al., 2001). Exonuclease 1 (NEB) is added to remove excess unbound detection probes. The cDNA is PCR amplified and then is quantified. The cDNA is fragmented and amplified for sequencing with the Nextera XT DNA sample prep kit (Illumina) using custom primers that enabled the specific amplification of only the 3′ ends (Macosko et al., 2015).

Alternatively, library preparation is based on the CEL-Seq protocol with minor modifications (Jaitin et al., 2014). The workflow of DNA library preparation is summarized as follows: reverse transcription (RT)→Exonuclease I→SPRI purification (SPRIP)→Second Strand Synthesis (SSS)→SPRIP→T7 in vitro transcription linear amplification→SPRIP→RNA Fragmentation→SPRIP→primer ligation→RT→library enrichment PCR (Klein et al., 2015).

Alternatively, detection probes containing a poly dT sequence capture the polyA tail of the mRNA. The mRNA is reverse transcribed to cDNA. Exonuclease 1 (NEB) is added to remove excess unbound detection probes. The cDNA is first PCR amplified using a gene specific primer and a primer against the universal primer sequence of the detection probe. A second PCR amplifies the first PCR products using a nested gene specific primer flanked by an Illumina sequencing primer and a primer against the universal primer sequence of the detection probe flanked by an Illumina sequencing primer. The third PCR adds P5 and P7 and a sample index to turn PCR products into an Illumina sequencing library (Fan et al., 2015).

Alternatively, detection probes containing a poly dT sequence capture the polyA tail of the mRNA. The mRNA is reverse transcribed to cDNA. Exonuclease 1 (NEB) is added to remove excess unbound detection probes. Alternatively, a SPRI cleanup is added to further purify the cDNA from unbound detection probes. A random-primed Klenow extension is used for second strand synthesis. Alternatively, rather than using Klenow another DNA polymerase is used for second strand synthesis. The random hexamer used for priming in the Klenow extension is flanked with a universal Illumina sequencing primer 1 sequence. The cDNA is PCR amplified using a primer against the universal Illumina sequencing primer 1 sequence flanked by the Illumina P5 or P7 adapter sequence and a primer against the universal primer sequence of the detection probe (that includes Illumina sequencing primer 2 sequence) flanked by the Illumina P5 or P7 adapter sequence to turn PCR products into an Illumina sequencing library (Derti et al., 2012).

Alternatively, if an Illumina sequencer is not used to sequence the RNA-seq libraries, the previously discussed methods in this example are modified so that the libraries are compatible with the sequencer used.

### Example 4: Methods to Fabricate Detection Probes for RNA and Protein Molecules

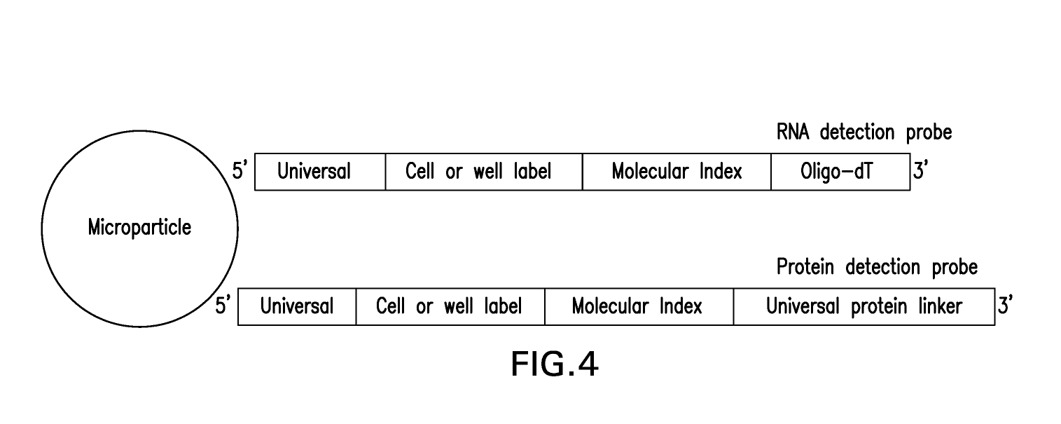

Microparticles synthesized with various methods for the detection of mRNA that include a poly-T sequence to bind to the poly-A tail of mRNA, a unique well/cell barcode sequences and unique molecular identifier sequences that enable digital counting of transcripts have been demonstrated in literature (Bose et al., 2015; Fan et al., 2015; Klein et al., 2015; Macosko et al., 2015). In addition to a poly-T sequence that binds to mRNA a complementary nucleotide sequence that binds to the nucleotide sequence of the protein target probe is added to enable simultaneous digital counting of RNA and protein molecules. So each microparticle consists of two different oligonucleotide sequences (A and B) composed of four parts where the first three parts are the same and the last part is different (FIG. 4): (1) a constant sequence (identical on all primers and beads) for use as a priming site for downstream PCR and sequencing; (2) a “cell or well barcode” (identical across all the primers on the surface of any one bead, but different from the cell barcodes on other beads); (3) a Unique Molecular Identifier (UMI) (different on each primer, to identify PCR duplicates and to enable digital counting of RNA and protein molecules) (Kivioja et al., 2012); and (4) for oligonucleotide sequence A, an oligo-dT sequence for capturing polyadenylated mRNAs and priming reverse transcription; and for oligonucleotide sequence B a complementary sequence to the universal protein linker nucleotide sequence. This oligonucleotide sequence B consists of DNA, RNA, or a combination of DNA and RNA nucleotides.

Barcoded Hydrogel microspheres (BHM) are synthesized using microfluidic emulsification of acrylamide:bis-acrylamide solution supplemented with acrydite-modified DNA primer, which is incorporated into the hydrogel mesh upon acrylamide polymerization. After polymerization, the BHMs are released from droplets, washed several times and processed by split-pool synthesis for combinatorial barcoding. To prepare barcoded primers on the hydrogel microspheres a multi-step enzymatic extension reaction is used. Each BHM carries ˜109-1013 covalently coupled, photo-releasable or cleavable primers. Doing the split and pool steps twice in 384 well format gives 384×384=147,456 barcodes which allows random labeling 3,000 cells with 99% unique labeling. The numbers of barcodes are increased in a straightforward manner by increasing the number of split pool steps and/or increasing from 384 to 1536 well plates (Klein et al., 2015). In the last step of the split and pool barcoding process a nucleotide sequence that consists of a complementary sequence to the extended BHM primer, a unique sequence for the well, and either a poly-T sequence or a complementary universal protein linker nucleotide sequence is added in equimolar concentrations. This results in the BHM having two distinct oligonucleotides, one with a poly-T sequence at the end to bind to the poly-A tail of mRNA and the other with a complementary universal protein linker nucleotide sequence that is complementary to the protein target probe.

Alternatively a “split-and-pool” DNA synthesis strategy is used to generate massive numbers of beads, each with a distinct barcode. Beads are functionalized and used as a solid support for reverse-direction phosphoramidite synthesis (5′ to 3′). A pool of millions of beads is divided into four equally sized groups. Then a different DNA base (dA, dG, dC, or dT) is added to each. After each split-and-pool phosphoramidite synthesis cycle, beads are then re-pooled, mixed, and re-split at random into another four groups (equal portions based on mass), and then a different DNA base (dA, dG, dC, or dT) is added to each of the four new groups. This process is repeated 10-15 times depending on the total number of unique barcoded sequences desired (4̂10-4̂15=1,048,576-1,073,741,824). After 12 cycles of split-and pool DNA synthesis, the primers on any given bead possess the same one of 412=16,777,216 possible 12-bp barcodes, but different bead have different sequences. The entire bead pool then undergoes eight rounds of degenerate oligonucleotide synthesis to generate the UMI on each oligonucleotide (Macosko et al., 2015). Then, a common linker sequence is synthesized on the 3′ end of all oligonucleotides on all beads. Finally, equimolar amounts of oligonucleotides bearing 1) the complement to the common linker and an oligo-dT sequence (T30) and 2) the complement to the common linker and the complement to the detection probe universal protein linker nucleotide sequence are added and annealed to the beads. Enzymatic polymerization extends the common linker sequence on the bead with the oligo-dT sequence or the universal protein linker nucleotide sequence. After enzymatic polymerization the beads are pooled to derive the final library where each bead has a unique bead barcode (10-15 bp) and a sequence either complementary to the poly A tail of mRNA or the protein target probe universal linker nucleotide sequence.

Beads are synthesized using a combinatorial split-pool synthesis method similar to the one used in for CytoSeq with slight modifications (Fan et al., 2015). Beads functionalized with carboxyl groups are distributed into 96, 384, or 1536 well plates. Using carbodiimide chemistry, a 5 amine modified oligonucleotide bearing a universal PCR priming sequence, a unique cell or well label (8-15 bp), and a common linker were coupled to the beads in each well. Conjugated beads from all wells are then pooled and split into a second set of 96, 384, or 1536 wells for annealing to template oligonucleotides bearing the complement to the common linker, another cell or well label (8-15 bp), and a new common linker. After enzymatic polymerization, the beads are again pooled and split into a third set of 96, 384, or 1536 wells for annealing to equimolar concentrations of oligonucleotides bearing either: 1) oligo(dA) (15-30 bp) on the 5′ end or 2) the complement to the protein target probe universal linker nucleotide sequence, followed by a randomly synthesized sequence (8-15 bp) that serves as the unique molecular identifier, a third cell or well label (8-15 bp), and a complementary sequence to the second linker. After enzymatic polymerization, the beads were pooled to derive the final library. Each resulting bead is coated with 10̂7-10̂12 of oligonucleotides A and B of the same clonally represented cell label (if 96 well plates were used in each of the three steps then 96×96×96=884,736 or possible barcodes) and a molecular indexing diversity of 4̂8=65,536 if an 8 bp unique molecular identifier is used. The library size is increased exponentially by linearly increasing the diversity at each step of synthesis. For example, if three split and pool cycles were conducted in 384 well plates then the library size increases to 384×384×384=56,623,104 possible barcodes.

To apply this biochemical assay to single cells, pools of cell, or cell lysates that are isolated into microwells, tubes, 96, 384, or 1536 well plates, detection probes in solution are used and do not necessarily have to be fabricated on microparticles such as magnetic beads, polymer beads, or in soft hydrogels. For each sample two different oligonucleotides are synthesized by IDT (Integrated DNA Technologies); 1) oligonucleotide A which binds to the poly A tail of the mRNA and 2) oligonucleotide B which binds to the protein target probe nucleotide sequence. Both oligonucleotide sequences (A and B) are composed of four parts (FIG. 4): (1) a constant sequence for use as a priming site for downstream PCR and sequencing; (2) a cell or well barcode; (3) a Unique Molecular Identifier (UMI) (different on each primer, to identify PCR duplicates and to enable digital counting of RNA and protein molecules) (Kivioja et al., 2012); and (4) for oligonucleotide sequence A an oligo-dT sequence for capturing polyadenylated mRNAs and priming reverse transcription and for oligonucleotide sequence B a complementary sequence to the protein target probe universal linker nucleotide sequence.

### Example 5: Methods to Separate Cells with Unique Detection Probes

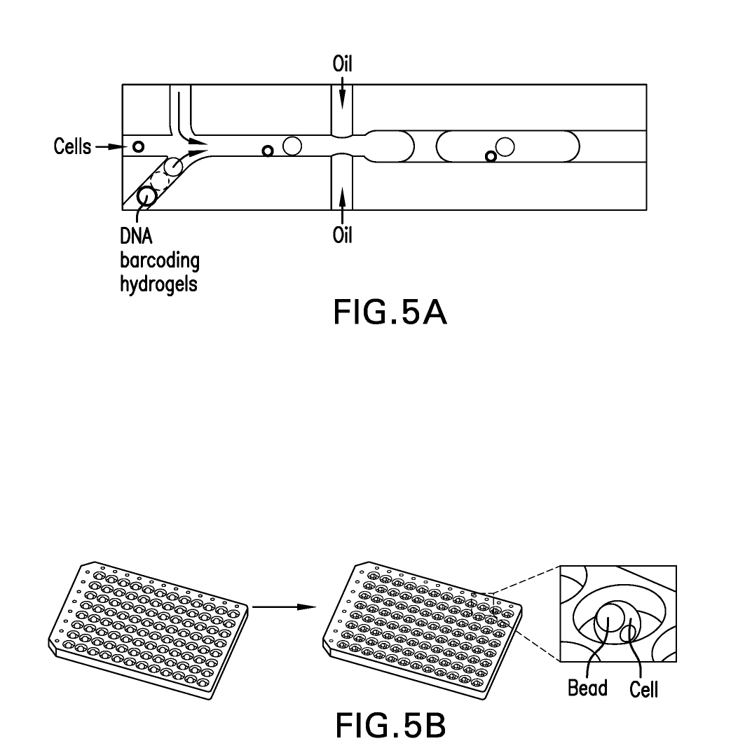

Single cells and microparticles are isolated in droplets using emulsion based methods (Klein et al., 2015; Macosko et al., 2015). A microfluidic device is used that flows two aqueous solutions across an oil channel to form droplets. One aqueous flow contains the microparticles with barcoded oligonucleotides suspended in a lysis buffer. The second flow contains a cell suspension. In an alternative approach a microfluidic device with four inlets for microparticles, cells, RT/lysis reagents, and carrier oil is used (FIG. 5A) (Klein et al., 2015). The concentrations of microparticles and cells in these two aqueous flows are optimized to maximize the number of droplets that contain one microparticle and one cell while minimizing the number of droplets that get two microparticles or two cells. In typical conditions, cells occupy only 10% of droplets, so two-cell events are rare and cell aggregates are minimized by passing cells through a strainer or by fluorescence-activated cell sorting (FACS). When deformable hydrogel microparticles are used they are packed closely and released regularly allowing nearly 100% hydrogel microparticle occupancy (Abate et al., 2009). Unlike conventional plates or valve-based microfluidics, the number of reaction chambers is not limited and droplets are intrinsically scalable which is advantageous for high throughput assays. The cross section of the microfluidics channels are designed to be large enough so that there is no cell size bias in capture.

Single cells are isolated using an array of microwells (FIG. 5B). (Bose et al., 2015; Fan et al., 2015; Love et al., 2006). Microwells are designed so that each microwell is a specific diameter (20-80 um) and depth (20 um-80 um). The number of cells deposited per well depended on the concentration of cells, the volume applied, the time allowed for settling, the size of the microwells and the size of the PDMS slab. The goal is to avoid depositing two cells in a well so the number of cells in suspension is adjusted so that only about 1 out of the 10 wells receives a cell and the cells settle into the wells by gravity. The microparticles containing the RNA and protein detection probes are then loaded onto the microwell array to saturation so that most wells are filled. The dimensions of the beads and wells are optimized to prevent double occupancy of microparticles (Fan et al., 2015).

Alternatively, single cells are captured in a microfluidic device based on size such as the Fluidigm C1system. The microfluidic chip contains an integrated fluidics circuit designed enabling to trap cells, lyse cells, and add appropriate assay reagents to each chamber that contains a cell.

Alternatively, single cells or pools of cells are isolated using fluorescence-activated cell sorting (FACS) into microwells, 96, 384, or 1536 well plates. The barcoded detection probes in solution or attached to a microparticle are added manually or via automation. Alternatively, barcoded detection probes attached to microparticles are added to single cells isolated in microwells, 96, 384, 1536 well plates via FACS to achieve one microparticle per well or microwell.

### REFERENCES

Abate, A. R., C. H. Chen, J. J. Agresti, and D. A. Weitz. 2009. Beating Poisson encapsulation statistics using close-packed ordering. Lab Chip. 9:2628-2631.
Agasti, S. S., M. Liong, V. M. Peterson, H. Lee, and R. Weissleder. 2012. Photocleavable DNA Barcode-Antibody Conjugates Allow Sensitive and Multiplexed Protein Analysis in Single Cells. J Am Chem Soc. 134:18499-18502.
Assarsson, E., M. Lundberg, G. Holmquist, J. Bjorkesten, S. B. Thorsen, D. Ekman, A. Eriksson, E. Rennel Dickens, S. Ohlsson, G. Edfeldt, A.-C. Andersson, P. Lindstedt, J. Stenvang, M. Gullberg, and S. Fredriksson. 2014. Homogenous 96-plex PEA immunoassay exhibiting high sensitivity, specificity, and excellent scalability. PloS one. 9:e95192.
Bose, S., Z. Wan, A. Carr, A. H. Rizvi, G. Vieira, D. Pe'er, and P. A. Sims. 2015. Scalable microfluidics for single-cell RNA printing and sequencing. Genome Biol. 16:120.
Brody, E. N., and L. Gold. 2000. Aptamers as therapeutic and diagnostic agents. J Biotechnol. 74:5-13.
Derti, A., P. Garrett-Engele, K. D. Macisaac, R. C. Stevens, S. Sriram, R. Chen, C. A. Rohl, J. M. Johnson, and T. Babak. 2012. A quantitative atlas of polyadenylation in five mammals. Genome Res. 22:1173-1183.
Dirksen, A., and P. E. Dawson. 2008. Rapid Oxime and Hydrazone Ligations with Aromatic Aldehydes for Biomolecular Labeling. Bioconjugate Chem. 19:2543-2548.
Dirksen, A., S. Dirksen, T. M. Hackeng, and P. E. Dawson. 2006a. Nucleophilic catalysis of hydrazone formation and transimination: Implications for dynamic covalent chemistry. J Am Chem Soc. 128:15602-15603.
Dirksen, A., T. M. Hackeng, and P. E. Dawson. 2006b. Nucleophilic catalysis of oxime ligation. Angew Chem Int Edit. 45:7581-7584.
Ellington, A. D., and J. W. Szostak. 1990. In vitro selection of RNA molecules that bind specific ligands. Nature. 346:818-822.
Famulok, M., J. S. Hartig, and G. Mayer. 2007. Functional aptamers and aptazymes in biotechnology, diagnostics, and therapy. Chem Rev. 107:3715-3743.
Fan, H. C., G. K. Fu, and S. P. Fodor. 2015. Expression profiling. Combinatorial labeling of single cells for gene expression cytometry. Science. 347:1258367.
Fredriksson, S., M. Gullberg, J. Jarvius, C. Olsson, K. Pietras, S. M. Gustafsdottir, A. Ostman, and U. Landegren. 2002. Protein detection using proximity-dependent DNA ligation assays. Nature biotechnology. 20:473-477.
Gold, L. 1995. Oligonucleotides as research, diagnostic, and therapeutic agents. J Biol Chem. 270:13581-13584.
Gong, H., I. Holcomb, A. Ooi, X. Wang, D. Majonis, M. A. Unger, and R. Ramakrishnan. 2016. Simple Method To Prepare Oligonucleotide-Conjugated Antibodies and Its Application in Multiplex Protein Detection in Single Cells. Bioconjug Chem.
Jaitin, D. A., E. Kenigsberg, H. Keren-Shaul, N. Elefant, F. Paul, I. Zaretsky, A. Mildner, N. Cohen, S. Jung, A. Tanay, and I. Amit. 2014. Massively Parallel Single-Cell RNA-Seq for Marker-Free Decomposition of Tissues into Cell Types. Science. 343:776-779.
Kasai, N., K. Kobayashi, S. Shioya, Y. Yoshikawa, F. Yotsumoto, S. Miyamoto, E. Mekada, and J. Enokizono. 2012. Soluble heparin-binding EGF-like growth factor (HB-EGF) detected by newly developed immuno-PCR method is a clear-cut serological biomarker for ovarian cancer. Am J Transl Res. 4:415-421.
Kivioja, T., A. Vaharautio, K. Karlsson, M. Bonke, M. Enge, S. Linnarsson, and J. Taipale. 2012. Counting absolute numbers of molecules using unique molecular identifiers. Nature Methods. 9:72-U183.
Klein, A. M., L. Mazutis, I. Akartuna, N. Tallapragada, A. Veres, V. Li, L. Peshkin, D. A. Weitz, and M. W. Kirschner. 2015. Droplet barcoding for single-cell transcriptomics applied to embryonic stem cells. Cell. 161:1187-1201.
Love, J. C., J. L. Ronan, G. M. Grotenbreg, A. G. van der Veen, and H. L. Ploegh. 2006. A microengraving method for rapid selection of single cells producing antigen-specific antibodies. Nat Biotechnol. 24:703-707.
Lundberg, M., A. Eriksson, B. Tran, E. Assarsson, and S. Fredriksson. 2011a. Homogeneous antibody-based proximity extension assays provide sensitive and specific detection of low-abundant proteins in human blood. Nucleic Acids Res. 39:e102.
Lundberg, M., S. B. Thorsen, E. Assarsson, A. Villablanca, B. Tran, N. Gee, M. Knowles, B. S. Nielsen, E. Gonzalez Couto, R. Martin, O. Nilsson, C. Fermer, J. Schlingemann, I. J. Christensen, H. J. Nielsen, B. Ekstrom, C. Andersson, M. Gustafsson, N. Brunner, J. Stenvang, and S. Fredriksson. 201 lb. Multiplexed homogeneous proximity ligation assays for high-throughput protein biomarker research in serological material. Mol Cell Proteomics. 10:M110 004978.
Macosko, E. Z., A. Basu, R. Satija, J. Nemesh, K. Shekhar, M. Goldman, I. Tirosh, A. R. Bialas, N. Kamitaki, E. M. Martersteck, J. J. Trombetta, D. A. Weitz, J. R. Sanes, A. K. Shalek, A. Regev, and S. A. McCarroll. 2015. Highly Parallel Genome-wide Expression Profiling of Individual Cells Using Nanoliter Droplets. Cell. 161:1202-1214.
Mocanu, M.-M., T. Varadi, J. Szollosi, and P. Nagy. 2011. Comparative analysis of fluorescence resonance energy transfer (FRET) and proximity ligation assay (PLA). Proteomics. 11:2063-2070.
Soderberg, O., M. Gullberg, M. Jarvius, K. Ridderstrale, K. J. Leuchowius, J. Jarvius, K. Wester, P. Hydbring, F. Bahram, L. G. Larsson, and U. Landegren. 2006. Direct observation of individual endogenous protein complexes in situ by proximity ligation. Nature Methods. 3:995-1000.
Tuerk, C., and L. Gold. 1990. Systematic evolution of ligands by exponential enrichment: RNA ligands to bacteriophage T4 DNA polymerase. Science (New York, N.Y.). 249:505-510.
Ullal, A. V., V. Peterson, S. S. Agasti, S. Tuang, D. Juric, C. M. Castro, and R. Weissleder. 2014. Cancer Cell Profiling by Barcoding Allows Multiplexed Protein Analysis in Fine-Needle Aspirates. Sci Transl Med. 6.
Zhu, Y. Y., E. M. Machleder, A. Chenchik, R. Li, and P. D. Siebert. 2001. Reverse transcriptase template switching: a SMART approach for full-length cDNA library construction. Biotechniques. 30:892-897.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method for detecting a plurality of target proteins and mRNA in a cell or cell lysate comprising:
a. contacting a cell or cell lysate with
1) a plurality of protein target probes, wherein each target probe in the plurality comprises:
i. a protein-binding molecule;
ii. a target nucleotide sequence comprising a primer for a polymerase chain reaction (PCR), a protein identification sequence that identifies the protein-binding molecule, a linker sequence that hybridizes to a sequence in the detection probe;
iii. a linker between the protein-binding molecule and the target nucleotide sequence; and
2) a plurality of microparticles comprising a plurality of detection probes comprising:
i. a protein detection nucleotide sequence comprising a primer for a polymerase chain reaction (PCR), a cell identification sequence that identifies the cell, a unique molecular identifier (UMI), and a complimentary linker sequence that hybridizes to the linker sequence in the target probe;
ii. an mRNA detection nucleotide sequence comprising a primer for a polymerase chain reaction (PCR), an cell identification sequence that identifies the cell, a unique molecular identifier, and polydT;
b. allowing the target nucleotide sequence to hybridize to the protein detection nucleotide sequence and the mRNA to hybridize to the mRNA detection nucleotide sequence in the microparticle;
c. conducting reverse transcription and/or polymerase extension to generate
i. a first analyte sequence comprising the primer, cell identification sequence, unique molecular identifier, and complimentary linker sequence in the detection probe; and protein identification sequence and the primer from the protein target probe;
ii. a second analyte sequence comprising the primer, cell identification sequence, unique molecular identifier, and polydT in the detection probe; and cDNA of the mRNA;
d. enriching the analyte sequences from unbound target and detection nucleotide sequences in the sample;
e. detecting signals from the analyte sequences based on PCR amplification and sequencing, wherein the signals are distinguishable for each protein and mRNA.

2. A method for detecting a plurality of target proteins and mRNA in a single cell:
a. contacting a single cell with a plurality of protein target probes, wherein each target probe in the plurality comprises:
1) a plurality of protein target probes, wherein each target probe in the plurality comprises:
i. a protein-binding molecule;
ii. a target nucleotide sequence comprising a primer for a polymerase chain reaction (PCR), a protein identification sequence that identifies the protein-binding molecule, a linker sequence that hybridizes to a sequence in the detection probe;
iii. a linker between the protein-binding molecule and the target nucleotide sequence; and
b. forming an emulsion droplet or microwell comprising the single cell and a microparticle comprising a plurality of detection probes comprising:
i. a protein detection nucleotide sequence comprising a primer for a polymerase chain reaction (PCR), a cell identification sequence that identifies the cell, a unique molecular identifier, and a complimentary linker sequence that hybridizes to the linker sequence in the target probe;
ii. an mRNA detection nucleotide sequence comprising a primer for a polymerase chain reaction (PCR), a cell identification sequence that identifies the cell, a unique molecular identifier, and polydT;
c. allowing the target nucleotide sequence to hybridize to the protein detection nucleotide sequence and the mRNA to hybridize to the mRNA detection nucleotide sequence in the microparticle;
d. conducting reverse transcription and/or polymerase extension to generate
i. a first analyte sequence comprising the primer, cell identification sequence, unique molecular identifier, and complimentary linker sequence in the detection probe; and
protein identification sequence and the primer from the protein target probe;
ii. a second analyte sequence comprising the primer, cell identification sequence, unique molecular identifier, and polydT in the detection probe; and cDNA of the mRNA;
e. enriching the analyte sequences from unbound target and detection nucleotide sequences in the sample;
f. detecting signals from the analyte sequences based on PCR amplification and sequencing, wherein the signals are distinguishable for each protein and mRNA

3. The method of claim 1, wherein the protein-binding molecule is an antibody or an aptamer.

4. The method of claim 1, wherein the linker is cleavable.

5. The method of claim 1, wherein the linker sequence that hybridizes to the detection probe is universal for the target probe.

6. The method of claim 1, wherein the primer comprises a common sequence to enable PCR amplification.

7. The method of claim 1, wherein the protein identification sequences have a length of about 5-20 nucleotides.

8. The method of claim 1, wherein the protein identification nucleotide sequences have a length of about 10 nucleotides.

9. The method of claim 1, wherein the cell identification sequences have a length of about 5-20 nucleotides.

10. The method of claim 1, wherein the cell identification sequences have a length of about 10-15 nucleotides.

11. The method of claim 1, wherein the UMI sequences have a length of about 4-20 nucleotides.

12. The method of claim 1, wherein the polydT sequences have a length of about 20-40 nucleotides.

13. The method of claim 1, wherein the microparticle comprises a polymer bead, magnetic bead, hydrogel microsphere, or resin that has the first and mRNA detection nucleotide sequence bound to or encapsulated in the microparticle.

14. A kit for multiplexed detection of a plurality of proteins and mRNA from a sample comprising
1) a plurality of protein target probes, wherein each target probe in the plurality comprises:
i. a protein-binding molecule;
ii. a target nucleotide sequence comprising a primer for a polymerase chain reaction (PCR), a protein identification sequence that identifies the protein-binding molecule, a linker sequence that hybridizes to a sequence in the detection probe;
iii. a linker between the protein-binding molecule and the target nucleotide sequence; and
2) a plurality of microparticles comprising a plurality of detection probes comprising:
i. a protein detection nucleotide sequence comprising a primer for a polymerase chain reaction (PCR), a cell identification sequence that identifies the cell, a unique molecular identifier, and a complimentary linker sequence that hybridizes to the linker sequence in the target probe; and
ii. an mRNA detection nucleotide sequence comprising a primer for a polymerase chain reaction (PCR), a cell identification sequence that identifies the cell, a unique molecular identifier, and polydT.
